(19) United States
(12) Patent Application Publication
(10) Pub. No.: US 2009/0117183 A1
(43) Pub. Date: May 7, 2009

(54) ORAL CONTRACEPTIVE CONTAINING A GESTAGEN AND AN ESTROGEN COMBINED WITH PHARMACEUTICALLY ACCEPTABLE AUXILIARY AGENTS AND/OR EXCIPIENTS, BUT NOT CONTAINING LACTOSE, AND METHOD OF MAKING SAME

(76) Inventors: Sabine Fricke, Jena (DE); Manuela Pfeifer, Jena (DE); Claus Claussen, Jena (DE); Ralf Ladwig, Jena (DE); Beate Buerglen, Jena (DE)

Correspondence Address: Striker, Striker & Stenby, 103 East Neck Road, Huntington, NY, 11743, US

(21) Appl. No.: 12/258,737

(22) Filed: Oct. 27, 2008

Related U.S. Application Data
(60) Provisional application No. 60/985,443, filed on Nov. 5, 2007.

(51) Int. Cl. A61K 9/24; A61K 31/566; A61P 15/18

(52) U.S. Cl. 424/465; 514/170; 424/472

(57) ABSTRACT

The method produces a lactose-free oral contraceptive composition containing a combination of a gestagen and an estrogen together with one or more pharmaceutically acceptable auxiliary agents and/or excipients. The contraceptive composition is a tablet, powder, or capsule that contains the gestagen and estrogen, filler material such as microcrystalline cellulose and a binder such as hydroxypropylcellulose, but no lactose. Preferably the gestagen is dienogest, chlormadinone acetate, or levonorgestrel and the estrogen is ethinylestradiol, 17β-estradiol, or estradiol valerate. A method is provided for improving the prophylaxis of lactose intolerance in women taking oral contraceptives. The oral contraceptive preparations for a standard 28-day cycle or for long-term use contain at least 21 daily dose units of the gestagen and the estrogen in a low-dosage but without lactose and at most 7 daily dose units containing no active ingredient or a placebo.

### CROSS-REFERENCE

The invention claimed and described herein below is also described in U.S. Provisional Patent Application Ser. No. 60/985,443, filed on Nov. 5, 2007. The aforesaid U.S. Provisional Patent Application provides the basis for a claim of priority of invention for the invention claimed herein below under 35 U.S.C. 119 (e).

### BACKGROUND OF THE INVENTION

1. The Field of the Invention

The invention relates to the use of a gestagen in combination with an estrogen and one or more pharmaceutically acceptable auxiliary agents/excipients without any lactose for production of a pharmaceutical preparation for lactose-free oral contraception. A method is provided for improving the prophylaxis for lactose intolerance and possibly also contributing toward an improvement in terms of costly examinations for lactose tolerance.

2. The Description of the Related Art

Lactose intolerance, also referred to as milk sugar intolerance, is a disorder that affects about 10 million people in Germany. Typical symptoms of lactose intolerance are, in the order of severity, abdominal pain, fullness feeling, bloating, nausea and diarrhea. Basically, one differentiates between genetically induced lactose intolerance resulting from, for example, a congenital enzyme defect, and acquired lactose intolerance the cause of which is still virtually unknown. The diagnosis of lactose intolerance requires an H₂-breathing test or a biopsy of the small intestine. In the small intestine lactose, a disaccharide and the carbohydrate present in milk, is split into the individual sugars, then taken up in the mucosal cells and transported further in the blood. If, because of the absence or reduced lactase activity, milk sugar ends up in the colon unsplit or only partly split, it is decomposed into lactic and acetic acid, carbon dioxide, hydrogen and methane by bacteria that have settled there. As a result, an osmotic pressure is created which brings about increased intestinal peristalsis with diarrhea. At the same time, the gases generated in the intestine lead to bloating or cramps. Depending on the severity of the lactose intolerance, the affected persons are advised to eat a lactose-free or low-lactose diet (<3 mg of lactose/day). A healthy adult takes in 20 to 30 g of lactose daily in a properly balanced diet. 100 g of cow milk contains about 5 g of lactose. Different kinds of bread and baked goods, sausage types, butter and margarine, chocolate and sweetener tablets contain lactose for technological reasons. Also recommended for therapy are lactase-containing enzyme preparations that are to be taken together with food in order to stimulate milk sugar splitting.

U.S. Pat. No. 6,881,428 describes a method of producing lactose-free milk by adding an enzyme (lactase) to the milk.

It is known from pharmaceutical technology to produce oral contraceptives (OC) exclusively based on lactose. Because of its outstanding properties, lactose is particularly well suited as formulation filler. It provides formulations characterized by strength, good disintegration performance and good stability. In lactose granulate, the steroid hormones, alone or in combination, are very advantageously distributed over the individual particle size classes. De-mixing of granulates that would result in insufficiently uniform distribution of the active ingredients contained in the tablet cores is practically unknown. It may be assumed that in a lactose-based OC the active ingredient is uniformly distributed, even in the very low-dose range (at least 15 μg of ethinylestradiol per unit). Lactose itself can be readily granulated. The resulting granulate can be processed into tablets without any problems. Direct tableting, which could conceivable be done with a special lactose, has not been considered thus far, because of the non-uniform content of the active ingredients in the individual formulations and is not used in practice. Oral contraceptives with low doses of active ingredients have thus far been produced by granulation, followed by tableting and in most cases by a coating process.

WO 2005/030175 discloses a preparation containing norethisterone acetate and estradiol and cellulose binders and in which the pharmaceutical preparation given as an example contains up to 45 wt. % of lactose.

WO 2005/030176 discloses a composition containing gestagens, cellulose binders and in which the pharmaceutical composition given as an example contains besides noresthisterone acetate and estradiol also up to 45 wt. % of lactose.

### SUMMARY OF THE INVENTION

It is an object of the present invention to provide a method of producing a lactose-free oral contraceptive composition for women with lactose intolerance or women who previously had not recognized their lactose intolerance.

It is another object of the present invention to provide a lactose-free oral contraceptive preparation for women with lactose intolerance or women who had not previously recognized their lactose intolerance.

It is a further object of the present invention to provide an improvement in the prophylaxis of lactose intolerance and if possible a reduction in the costs associated with the diagnosis and treatment of lactose intolerance in women who are taking oral contraceptives.

According to the invention these objects and others, which will be made more apparent herein after, are attained by a method of producing a lactose-free oral contraceptive composition comprising combining one or more pharmaceutically acceptable auxiliary agents and/or excipients, but not including lactose, with a gestagen and one or more estrogens, to form the lactose-free oral contraceptive composition.

In preferred embodiments of the method of producing the lactose-free oral contraceptive composition the gestagen is 17α-cyanomethyl-17-β-hydroxyestra-4,9-dien-3-one (dienogest) at a daily dose of 2.0 mg or 1.5 mg, chlormadinone acetate at a daily dose equivalent to the daily dose of dienogest, or levonorgestrel at a daily dose equivalent to the daily does of dienogest, and the estrogen is ethinylestradiol at a daily dose of 0.030 mg or 0.020 mg of 0.015 mg, estradiol at a daily dose equivalent to the daily dose of ethinylestradiol, or estradiol valerate at a daily dose equivalent to the daily dose of ethinylestradiol. Ethinylestradiol can also be present in the composition in the form of a clathrate.

Conceivably, other gestagens can also be used in the method of production of the lactose-free oral contraceptive preparation according to the invention. The method of production of the lactose-free oral contraceptives of the invention is based on the use of cellulose. The formulations are prepared by granulation, tableting or often by coating.

A method of producing of low-dose OC's that so far has not been practiced utilizes cellulose as filler and the granulation of the active ingredients together with a binder. The binder used in the method is preferably hydroxypropylcellulose (HPC). The HPC binder is preferably included in the core material in an amount of from 1 to 5 wt. %, based on the core material. However hypromellose, malto-dextrin, gelatin or starch paste can also be used as the binder. This has thus far not been considered, because celluloses with their highly restricted particle size range presented problems, especially in terms of active ingredient distribution.

According to the invention the aforesaid objects were also attained by selecting and optionally combining different kinds of microcrystalline cellulose (MCC) differing in bulk density, particle size and moisture content, such as AVICEL® PH 101, AVICEL® PH 102 or AVICEL® PH 112, or combinations of MCC and dibasic calcium phosphate or mannitol, while at the same time selecting an optimum ratio of binder to the total weight of the formulation (1-5 wt. % to 100 wt. %). Surprisingly, in this manner a uniform distribution of the, in most cases low-dosage, gestagen and estrogen in the individual granulate fractions and subsequently in the tablet was obtained. In the case of the very low-dose EE (at this time a minimum of 15 μg per tablet is possible), to achieve better distribution during the granulation process, the active ingredient can be sprayed onto a fluidized bed in the form of ethanolic ethinylestradiol.

In a preferred embodiment of the method of making the oral contraceptive composition the lactose-free orally administered tablet comprises a tablet core containing a part of a total amount of the gestagen to be released in a retarded manner and a coating around the tablet core, which contains a remaining part of the total amount of the gestagen to be released in a non-retarded manner and a total amount of the estrogen, which is to be released in a non-retarded manner. The gestagen is preferably dienogest and the estrogen is preferably ethinylestradiol. The tablet comprising the coated tablet core is advantageously prepared so that at least 10% and preferably 30% of the total amount of the gestagen dissolves from the tablet core in a retarded manner after more than 30 minutes as measured by a dissolution test using water at 37° C. as dissolution medium and a stirring velocity of 50 rpm.

The present invention also includes a method of producing a lactose-free pharmaceutical preparation for oral contraception, which includes the steps of:

a) preparing at least 21 daily dose units, each of which do not contain any lactose and contain a combination of a gestagen and an estrogen together with one or more pharmaceutically acceptable auxiliary agents and pharmaceutically acceptable excipients; and

b) preparing at most 7 daily dose units, each of which do not contain any lactose and each of which do not contain the combination of the gestagen and the estrogen or contain a placebo.

The lactose-free oral contraceptive composition can be in the form of a contraceptive preparation for a standard 28-day cycle with 21, 22, 23, 24, or 25 daily dose units each containing the gestagen and estrogen and with 7, 6, 5, 4, or 3 daily dose units containing a placebo.

The present invention also encompasses a lactose-free oral contraceptive composition made by the above-described method of producing the oral contraceptive composition.

### BRIEF DESCRIPTION OF THE DRAWING

The objects, features and advantages of the invention will now be illustrated in more detail with the aid of the following description of the examples, with reference to the accompanying figures in which:

FIGS. 1 and 2 are respective graphical illustrations of the time dependence of the percentage release of active ingredients from an example of the oral contraceptive preparation of the present invention, which does not contain any lactose, and the percentage release of active ingredients from a corresponding example of an oral contraceptive preparation of the prior art, which contains lactose in corresponding dissolution tests under different conditions.

### EXAMPLES

### Example 1

The active ingredient combination in example 1 is a combination of 2.0 mg of 17α-cyanomethyl-17-β-hydroxyestra-4,9-dien-3-one, also called dienogest, (DNG) and 0.030 mg of ethinylestradiol (EE).

### Example 2

The active ingredient combination in example 2 is a combination of 2 mg of chlormadinone acetate (CMA) and 0.030 mg of ethinylestradiol (EE).

### Example 3

The active ingredient combination in example 3 is a combination of 0.125 mg of levonorgestrel (LNG) and 0.030 mg of ethinylestradiol (EE).

### Example 4

The active ingredient combination in example 3 is a combination of 1.5 mg of dienogest (DNG) and 0.015 mg of ethinylestradiol (EE), of which 0.825 mg of the DNG and 0.015 mg of the EE are ingredients of a coating of the composition. The remainder of the DNG is contained in a core of the oral contraceptive composition.

The detailed compositions of examples 1 to 4 are presented in the following Table I.

TABLE I
COMPOSITIONS OF THE LACTOSE-FREE ORAL CONTRACEPTIVE
PREPARATIONS OF EXAMPLES 1 TO 4 OF THE INVENTION

| INGREDIENT | | | | EXAMPLE 4 |
|---|---|---|---|---|
| (CORE) | EXAMPLE 1 | EXAMPLE 2 | EXAMPLE 3 | (MR)* |
| Active | 2 mg of DNG & | 2 mg of DNG & | 0.125 mg of | 0.675 mg of DNG |
| ingredients | 0.03 mg of EE | 0.03 mg of EE | LNG &0.03 mg | |
| | | | of EE | |
| Filler | 43.52 mg of MCC | 43.52 mg of MCC | 46.395 mg of | 58.425 mg of MCC |
| | | | MCC | |
| Matrix former | — | — | — | 9 mg of hypromellose |
| Disintegrant | 1.95 mg of croscarmellose | 1.95 mg of croscarmellose | 1.95 mg of croscarmellose | 15 mg of corn |
| | Na | Na | Na | starch |
| Binder | 2 mg of | 2 mg of | 2 mg of | 6 mg of |
| | hydroxypropyl | hydroxypropyl | hydroxypropyl | maltodextrin |
| | cellulose | cellulose | cellulose | |
| Lubricant | 0.5 mg of | 0.5 mg of | 0.5 mg of | 0.9 mg of |
| | magnesium | magnesium | magnesium | magnesium |
| | stearate | stearate | stearate | stearate |

*Composition of the core only. The core of the oral contraceptive preparation of example 4 has a coating containing 0.825 mg of DNG and 0.015 mg of EE.

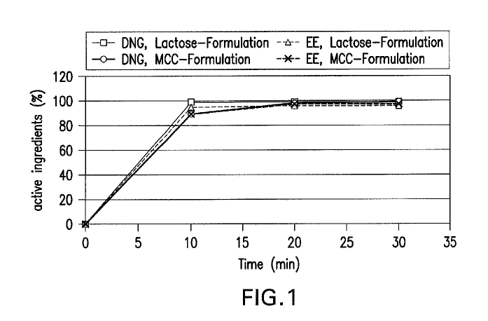

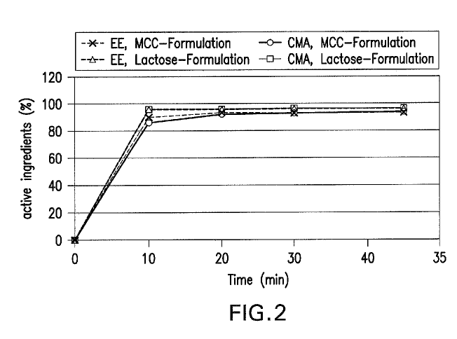

FIGS. 1 and 2 show comparisons of the time dependence of the percentage release of the active ingredients, DNG and EE, from oral contraceptive preparations of the present invention, which do not contain lactose, and corresponding conventional oral contraceptive preparations, which contain lactose.

In the case of the comparison shown in FIG. 1 both the oral contraceptive preparation of the present invention and the conventional preparation contained 2.0 mg of DNG and 0.030 mg of EE. The percentage release was measured in a dissolution test in a release apparatus using the paddling method with water at 37° C. as dissolution medium with a stirring velocity of 100 rpm. The comparison in FIG. 1 shows that the time dependence of the percentage active ingredient release of the lactose-free, cellulose-containing oral contraceptive preparation is substantially the same as that of the lactose-containing conventional preparation.

In the case of the comparison shown in FIG. 2 both the oral contraceptive preparation of the present invention and the conventional preparation contained 2.0 mg of CMA and 0.030 mg of EE. The percentage release was measured in a dissolution test in a release apparatus using the paddling method with sodium dodecyl sulfate at 37° C. as dissolution medium with a stirring velocity of 75 rpm. The comparison in FIG. 2 shows that the time dependence of the percentage active ingredient release of the lactose-free, cellulose-containing oral contraceptive preparation is substantially the same as that of the lactose-containing conventional preparation.

Table II shows a comparison of cellulose formulations differing in terms of active ingredient distribution in granulate and tablet core as a function of the type of cellulose and the quantity of binder. Uniform distribution of the active ingredients is established by determination of the active ingredient content of the individual screen fractions (fine, medium, coarse) and determination of the CUT of the tablet cores during the tableting process (beginning, middle, end).

TABLE II
CELLULOSE-BASED PREPARATIONS OF THE PRESENT
INVENTION WITH DIFFERENT ACTIVE INGREDIENT
DISTRIBUTIONS IN THEIR GRANULATE AND
TABLET CORE

| | Batch Nr. | Batch Nr. | Batch Nr. |
|---|---|---|---|
| COMPOSITION | 550907 | 621007 | 631007 |
| CMA | 2 mg | 2 mg | 2 mg |
| EE | 0.030 mg | 0.030 mg | 0.030 mg |
| AVICEL ® PH 101 | — | — | 36.02 mg |
| AVICEL ® PH 102 | 44.52 mg | 28.52 mg | — |
| AVICEL ® PH 112 | — | 15 mg | 7.5 mg |
| Croscarmellose Na | 1.95 mg | 1.95 mg | 1.95 mg |
| Magnesium stearate | 0.5 mg | 0.5 mg | 0.5 mg |
| DISTRIBUTION | | | |
| OF SCREEN | | | |
| FRACTIONS | | | |
| Fine fraction | CMA 125% | CMA 90.5% | CMA 99.4% |
| | EE 148% | EE 104.9% | EE 107.7% |
| Medium fraction | CMA 92% | CMA 114.9% | CMA 97.3% |
| | EE 93% | EE 118.5% | EE 96.9% |
| Course fraction | CMA 79% | CMA 125.9% | CMA 111.9% |
| | EE 52% | EE 116% | EE 102.5% |
| CORE CUT VALUES* | | | |
| START | CMA 2.75 | CMA 3.89 | CMA 2.27 |
| | EE 3.78 | EE 4.20 | EE 9.65 |
| MIDDLE | CMA 4.59 | CMA 7.15 | CMA 3.74 |
| | EE 5.33 | EE 8.59 | EE 3.01 |
| END | CMA 4.68 | CMA 3.58 | CMA 7.12 |
| | EE 4.52 | EE 6.71 | EE 9.24 |

*average values; target <15

While the invention has been illustrated and described as embodied in an oral contraceptive containing a gestagen and an estrogen combined with one or more pharmaceutically acceptable auxiliary agents and/or excipients, but not containing lactose, and method of making same, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A method of producing a lactose-free oral contraceptive composition containing a combination of a gestagen and an estrogen, said method comprising the step of combining said combination of said gestagen and said estrogen with at least one ingredient selected from the group consisting of pharmaceutically acceptable auxiliary agents and pharmaceutically acceptable excipients;
wherein said at least one ingredient does not include lactose; and
wherein said contraceptive composition contains said combination of said gestagen and said estrogen and said at least one ingredient selected from the group consisting of pharmaceutically acceptable auxiliary agents and pharmaceutically acceptable excipients.

2. The method as defined in claim 1, wherein said gestagen is dienogest, chlormadinone acetate, or levonorgestrel and wherein said estrogen is ethinyl-estradiol, 17-β-estradiol, or estradiol valerate.

3. The method as defined in claim 2, wherein said dienogest is present in said contraceptive composition in a daily dose equal to or less than 2 mg, said chlormadinone acetate is present in said contraceptive composition in a daily dose equivalent to said daily dose of said dienogest, or said levonorgestrel is present in said contraceptive composition in a daily dose equivalent to said daily dose of said dienogest.

4. The method as defined in claim 3, wherein said daily dose of said dienogest is 2 mg or 1.5 mg.

5. The method as defined in claim 2, wherein said ethinylestradiol is present in said contraceptive composition in a daily dose equal to or less than 0.030 mg, said estradiol is present in said contraceptive composition in a daily dose equivalent to said daily dose of said ethinylestradiol, or said estradiol valerate is present in said contraceptive composition in a daily dose equivalent to said daily dose of said ethinylestradiol.

6. The method as defined in claim 5, wherein said daily dose of said ethinylestradiol is 0.030 mg, 0.020 mg, or 0.015 mg.

7. The method as defined in claim 1, wherein said contraceptive composition is in the form of a tablet, coated tablet, sugar-coated tablet, capsule, or powder.

8. The method as defined in claim 1, further comprising preparing a tablet core containing a part of a total amount of said gestagen in said contraceptive composition that is to be released in a retarded manner and a coating around said tablet core, said coating containing a remainder of said total amount of said gestagen that is to be released in a non-retarded manner and a total amount of said estrogen, which is to be released in a non-retarded manner.

9. The method as defined in claim 8, wherein at least 10% of said total amount of said gestagen dissolves out of said tablet core in said retarded manner after more than 30 minutes as measured by a dissolution test using water at 37° C. as dissolution medium and a stirring velocity of 50 rpm.

10. The method as defined in claim 8, wherein at least 30% of said total amount of said gestagen dissolves out of said tablet core in said retarded manner after more than 30 minutes as measured by a dissolution test using water at 37° C. as dissolution medium and a stirring velocity of 50 rpm.

11. The method as defined in claim 8, wherein said gestagen is dienogest and said estrogen is ethinylestradiol.

12. The method as defined in claim 7, wherein said at least one ingredient comprises filler and a binder.

13. The method as defined in claim 12, wherein said filler is cellulose and said binder is selected from the group consisting of hydroxypropylcellulose, hypromellose, maltodextrin, gelatin and starch paste.

14. The method as defined in claim 12, wherein said at least one ingredient further comprises a disintegrant and a lubricant.

15. The method as defined in claim 14, wherein said disintegrant is croscarmellose sodium and said lubricant is magnesium stearate.

16. The method as defined in claim 1, wherein said contraceptive composition contains microcrystalline cellulose as filler and from 1 to 5 wt. % of hydroxypropyl-cellulose as a binder.

17. The method as defined in claim 7, further comprising granulating to form said powder or tableting with or without coating to form said tablet.

18. The method as defined in claim 17, further comprising spraying an ethanolic estradiol onto a fluidized bed during the granulating to make low-dose formulations.

19. A method of producing a lactose-free pharmaceutical preparation for oral contraception, said method comprising the steps of:
a) preparing at least 21 daily dose units each containing a combination of a gestagen and an estrogen together with at least one ingredient that is not lactose and is selected from the group consisting of pharmaceutically acceptable auxiliary agents and pharmaceutically acceptable excipients; and
b) preparing at most 7 daily dose units that do not contain said combination of said gestagen and said estrogen or that contain a placebo;
wherein each of said at least 21 daily dose units does not contain lactose and each of said at most 7 daily dose units does not contain lactose;
whereby said lactose-free pharmaceutical preparation comprises said at least 21 daily dose units each containing said combination of said gestagen and said estrogen together with said at least one ingredient selected from the group consisting of said pharmaceutically acceptable auxiliary agents and said pharmaceutically acceptable excipients and said at most 7 daily dose units that do not contain said combination of said gestagen and said estrogen or that contain said placebo.

20. The method as defined in claim 19, wherein said lactose-free pharmaceutical preparation comprises 21, 22, 23, 24, or 25 of said daily dose units each containing said combination of said gestagen and said estrogen together with said at least one ingredient selected from the group consisting of said pharmaceutically acceptable auxiliary agents and said pharmaceutically acceptable excipients and 7, 6, 5, 4, or 3 of said daily dose units that do not contain said combination of said gestagen and said estrogen or that contain said placebo.

21. The method as defined in claim 19, wherein said lactose-free pharmaceutical preparation comprises n×21 of said daily dose units each containing said combination of said gestagen and said estrogen together with said at least one ingredient selected from the group consisting of said pharmaceutically acceptable auxiliary agents and said pharmaceutically acceptable excipients, and wherein n=2, 3, 4, 5, 6, 7, 8, 9, 10, 11, 12, 13, 14, 15, 16, and 17.

22. The method as defined in claim 21, wherein said lactose-free pharmaceutical preparation contains 3, 4, 5, 6, or 7 of said daily dose units that do not contain said combination of said gestagen and said estrogen or that contain said placebo.

23. The method as defined in claim 22, wherein said lactose-free pharmaceutical preparation contains 84 of said daily dose units each containing said combination of said gestagen and said estrogen together with said at least one ingredient selected from the group consisting of said pharmaceutically acceptable auxiliary agents and said pharmaceutically acceptable excipients and 7 of said daily dose units that do not contain said combination of said gestagen and said estrogen or that contain said placebo, so that a total number of cycle days per year is 4×(m×21+7), wherein m=4.

24. A lactose-free oral contraceptive composition in the form of a tablet, coated tablet, sugar-coated tablet, capsule, or powder, wherein said oral contraceptive composition does not contain any lactose and contains
a combination of a gestagen and an estrogen; and
at least one ingredient selected from the group consisting of pharmaceutically acceptable auxiliary agents and pharmaceutically acceptable excipients.

25. The oral contraceptive composition as defined in claim 24, wherein dienogest is present as said gestagen in a daily dose equal to or less than 2 mg, chlormadinone acetate is present as said gestagen in a daily dose equivalent to said daily dose of said dienogest, or levonorgestrel is present as said gestagen in a daily dose equivalent to said daily dose of said dienogest.

26. The oral contraceptive composition as defined in claim 24, wherein ethinylestradiol is present as said estrogen in a daily dose equal to or less than 0.030 mg, estradiol is present as said estrogen in a daily dose equivalent to said daily dose of said ethinylestradiol, or estradiol valerate is present as said estrogen in a daily dose equivalent to said daily dose of said ethinylestradiol.

27. The oral contraceptive composition as defined in claim 24, which comprises a tablet core containing a part of a total amount of said gestagen to be released in a retarded manner and a coating around said tablet core, said coating containing a remainder of said total amount of said gestagen that is to be released in a non-retarded manner and a total amount of said estrogen, which is to be released in a non-retarded manner.

28. The oral contraceptive composition as defined in claim 24, wherein said at least one ingredient comprises filler and a binder.

29. The oral contraceptive composition as defined in claim 28, wherein said filler is cellulose and said binder is selected from the group consisting of hydroxypropyl-cellulose, hypromellose, maltodextrin, gelatin and starch paste.

30. The oral contraceptive composition as defined in claim 28, wherein said at least one ingredient further comprises a disintegrant and a lubricant.

31. The oral contraceptive composition as defined in claim 30, wherein said disintegrant is croscarmellose sodium and said lubricant is magnesium stearate.

32. The oral contraceptive composition as defined in claim 24, wherein said at least one ingredient comprises cellulose as filler and from 1 to 5 wt. % of hydroxypropylcellulose as binder.
